(19) United States
(12) Patent Application Publication
(10) Pub. No.: US 2019/0099252 A1
(43) Pub. Date: Apr. 4, 2019

(54) SURGICAL MESH HAVING INGROWTH-PREVENTING COATING ON ONE SIDE THEREOF, AND METHOD FOR MAKING THE SAME

(71) Applicant: Grant Technologies LLC, Chicago, IL (US)

(72) Inventors: Eric Nelson, Boise, ID (US); John W. Huelskamp, Chicago, IL (US); William Wiecek, Chicago, IL (US)

(21) Appl. No.: 16/144,295

(22) Filed: Sep. 27, 2018

Related U.S. Application Data
(60) Provisional application No. 62/567,335, filed on Oct. 3, 2017.

(51) Int. Cl. A61F 2/00; A61L 31/14

(52) U.S. Cl. CPC A61F 2/0063; D04B 21/10; D10B 2509/08; A61L 31/146

(57) ABSTRACT

A barrier surgical mesh, the barrier surgical mesh comprising: a matrix of fibers, the matrix comprising a first surface and a second surface; and a tissue ingrowth-preventing material applied to the second surface of the matrix; wherein the tissue ingrowth-preventing material penetrates a controllable amount part way through the matrix.

### REFERENCE TO PENDING PRIOR PATENT APPLICATION

This patent application claims benefit of pending prior U.S. Provisional Patent Application Ser. No. 62/567,335, filed Oct. 3, 2017 by Grant Technologies LLC and Eric Nelson et al. for SURGICAL MESH HAVING INGROWTH-PREVENTING COATING ON ONE SIDE THEREOF, AND METHOD FOR MAKING THE SAME (Attorney's Docket No. BGMEDICAL-5 PROV), which patent application is hereby incorporated herein by reference.

### FIELD OF THE INVENTION

This invention relates to medical procedures and apparatus in general, and more particularly to medical procedures and apparatus for repairing soft tissue defects such as inguinal and abdominal wall hernias and the like.

### BACKGROUND OF THE INVENTION

In the reconstruction of soft tissue defects in humans and animals, such as in abdominal hernia repairs, surgical mesh is often used to reinforce the soft tissue defect so as to reinforce the abdominal wall, facilitate healing and prevent subsequent defect recurrence. More particularly, when performing a closure of a soft tissue defect in an anatomical wall (e.g., to repair an abdominal hernia), it is common to secure a surgical mesh to the soft tissue at the edge of the soft tissue defect, with some overlap between the surgical mesh and the soft tissue, so as to increase the healed strength of the surgical repair. The surgical mesh is typically secured to the soft tissue adjacent to the soft tissue defect with suture or tacks, a type of fixation device.

It is common to form the surgical mesh out of a knitted/woven material or a non-knitted/non-woven material.

More particularly, and looking first at FIG. 1, there is shown a knitted/woven surgical mesh 5 which generally comprises a porous knitted/woven material 10 for effecting a tissue repair. Knitted/woven surgical mesh 5 generally comprises a first surface 12 which is intended to be adhered to tissue (e.g., to an abdominal wall, in the case of a hernia mesh) and a second surface 13 which is intended to face away from the tissue to which the surgical mesh is adhered (e.g., to face toward an abdominal cavity, in the case of a hernia mesh).

Looking next a FIG. 2, there is shown a non-knitted/non-woven surgical mesh 15 which generally comprises a porous non-knitted/non-woven material 20. Non-knitted/non-woven surgical mesh 15 generally comprises a first surface 22 which is intended to be adhered to tissue (e.g., to an abdominal wall, in the case of a hernia mesh) and a second surface 23 which is intended to face away from the tissue to which the surgical mesh is adhered (e.g., to face toward an abdominal cavity, in the case of a hernia mesh). By way of example but not limitation, non-knitted/non-woven surgical mesh 15 may comprise a matrix of synthetic fibers (e.g., polypropylene) fused together (e.g., at 25) by a point-bonding fusing process (e.g., by point-bonding melting) so as to form a composite structure. In this respect it will be appreciated that the compressive heat process typically leaves a plurality of regularly-spaced recesses 30 which extend partially into, but not all of the way through, the thickness of the non-knitted/non-woven surgical mesh. It should be appreciated that these recesses 30 extending partially into, but not all the way through, the thickness of the non-knitted/non-woven surgical mesh constitute only a small percentage of the total non-knitted/non-woven mesh area. Note that the non-knitted/non-woven surgical mesh material disposed at recesses 30 (e.g., at 25) typically has a higher density than the remainder of the non-knitted/non-woven mesh material, since the non-knitted/non-woven surgical mesh material has been compacted and melted in this region of the non-knitted/non-woven surgical mesh.

In practice, it has been found that it is often desirable to configure the surgical mesh so that it will encourage tissue ingrowth into one side of the surgical mesh (e.g., the abdominal wall side of the surgical mesh) while preventing tissue ingrowth into the opposite side of the surgical mesh (e.g., the abdominal cavity side of the surgical mesh). To this end, it is common to apply an ingrowth-preventing coating formed out of a permanent or resorbable non-porous flexible material (e.g., an elastomer such as silicone or urethane or a flexible resorbable material) to one side of the surgical mesh so that the ingrowth-preventing coating closes off the pores of the mesh (e.g., in the case of a knitted/woven mesh, the pores of the knitted/woven material forming the mesh, and in the case of a non-knitted/non-woven mesh, the recesses 30 of the non-knitted/non-woven material forming the mesh and the pores between the polypropylene fibers). See, for example, FIG. 3, where an ingrowth-preventing coating 35 (e.g., a permanent or resorbable non-porous flexible coating such as silicone, urethane, a flexible resorbable material, etc.) is applied to second surface 13 of knitted/woven surgical mesh 5 (e.g., the abdominal cavity side of the knitted/woven surgical mesh), in order to prevent adhesion of internal organs to the surgical mesh and thus creating a barrier surgical mesh. See also, for example, FIG. 4, where an ingrowth-preventing coating 35 (e.g., a permanent or resorbable non-porous flexible coating such as silicone, urethane, a flexible resorbable material, etc.) is applied to second surface 23 of non-knitted/non-woven surgical mesh 15 (e.g., the surface of the non-knitted/non-woven surgical mesh 15 which faces the abdominal cavity) so that the ingrowth-preventing coating closes off the recesses 30 and pores of the non-knitted/non-woven mesh and thus creating a barrier surgical mesh.

In practice, ingrowth-preventing coating 35 (e.g., a permanent or resorbable non-porous flexible coating such as silicone, urethane, a flexible resorbable material, etc.) is commonly applied to knitted/woven surgical mesh 5, and/or to non-knitted/non-woven surgical mesh 15, using a mechanical compression process such as calendaring, where the ingrowth-preventing coating is applied to one side of the surgical mesh (i.e., second surface 13 of knitted/woven surgical mesh 5 and/or second surface 23 of non-knitted/non-woven surgical mesh 15) and then the surgical mesh is passed through rollers and exposed to a curing process in order to bond the ingrowth-preventing coating to the surgical mesh.

While the mechanical compression process is effective in applying/fixing ingrowth-preventing coating 35 (e.g., a permanent or resorbable non-porous flexible coating such as silicone, urethane, a flexible resorbable material, etc.) to the surgical mesh, it has been found that the mechanical compression process causes the ingrowth-preventing coating to penetrate into the surgical mesh in an irregular fashion, i.e., ingrowth-preventing coating 35 penetrates irregularly into porous knitted/woven material 10 of knitted/woven surgical mesh 5 (FIG. 3) and/or into the porous non-knitted/non-woven material 20 of non-knitted/non-woven surgical mesh 15 (FIG. 4). In some cases, and as seen in FIGS. 3 and 4, ingrowth-preventing coating 35 (e.g., a permanent or resorbable non-porous flexible coating such as silicone, urethane, a flexible resorbable material, etc.) may penetrate all the way through the surgical mesh (e.g., such as is shown at 40) to first surface 12 of surgical mesh 5 (i.e., the surface of knitted/woven surgical mesh 5 which faces the abdominal wall) and/or to first surface 22 of non-knitted/non-woven surgical mesh 15 (i.e., the surface of non-knitted/non-woven surgical mesh 15 which faces the abdominal wall). Where this occurs, ingrowth-preventing coating 35 (e.g., a permanent or resorbable non-porous flexible coating such as silicone, urethane, a flexible resorbable material, etc.) prevents tissue ingrowth on the abdominal wall side of the surgical mesh (i.e., tissue ingrowth into first surface 12 of knitted/woven surgical mesh 5 and/or tissue ingrowth into first surface 22 of non-knitted/non-woven surgical mesh 15), reducing the integration of the surgical mesh into the surrounding tissues and potentially leading to hernia recurrence.

Thus there is a need for a new and improved approach for applying an ingrowth-preventing coating (which is permanent or resorbable, and adhesion-preventing) on one side of a surgical mesh (e.g., on the abdominal cavity side of a surgical mesh) while ensuring that the ingrowth-preventing coating does not penetrate through to the other side of the surgical mesh (e.g., the abdominal wall side) of the surgical mesh and supports adjacent improved tissue integration at the desired locations.

### SUMMARY OF THE INVENTION

The present invention comprises a new and improved approach for applying an ingrowth-preventing coating (which is permanent or resorbable, and adhesion-preventing) on one side of a surgical mesh (e.g., on the abdominal cavity side of a surgical mesh) while ensuring that the ingrowth-preventing coating does not penetrate through to the other side of the surgical mesh (e.g., the abdominal wall side).

In one preferred form of the present invention, there is provided a surgical mesh, said surgical mesh comprising:

a matrix of fibers, said matrix comprising a first surface and a second surface; and

a tissue ingrowth-preventing material applied to said second surface of said matrix;

wherein said tissue ingrowth-preventing material penetrates only part way through said matrix.

In another preferred form of the present invention, there is provided a method of manufacturing a surgical mesh, said method comprising:

providing a matrix of fibers, said matrix comprising a first surface and a second surface; and

spraying a tissue ingrowth-preventing material onto said second surface of said matrix such that said tissue ingrowth-preventing material penetrates into said matrix part way, but not all of the way, through said matrix.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing an exemplary knitted/woven surgical mesh;

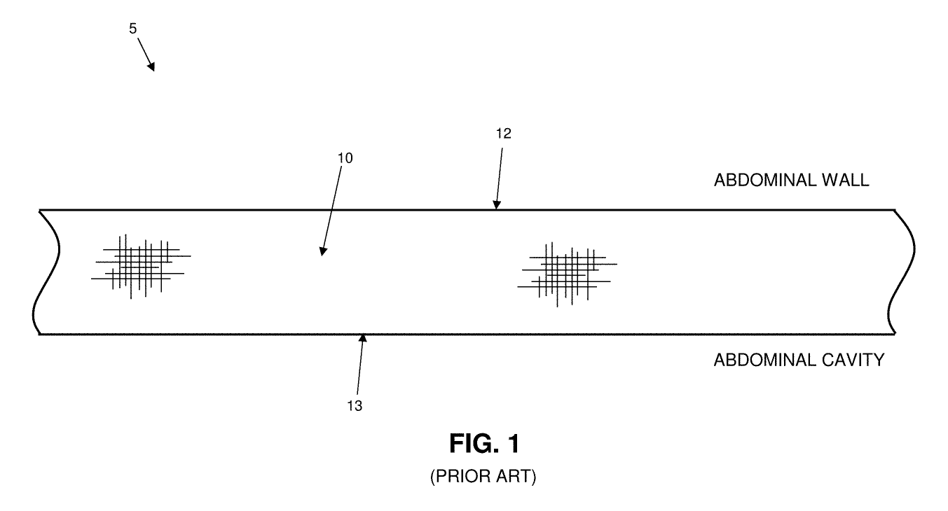

FIG. 2 is a schematic view showing an exemplary non-knitted/non-woven surgical mesh;

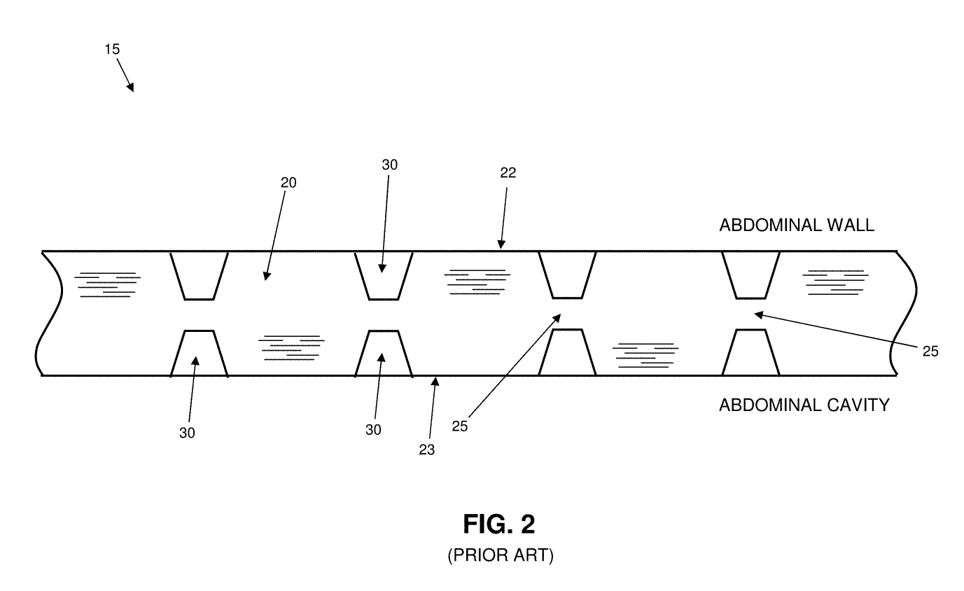

FIG. 3 is a schematic view showing a knitted/woven surgical mesh having an ingrowth-preventing coating on one side of the knitted/woven surgical mesh, wherein the ingrowth-preventing coating penetrates completely through the knitted/woven surgical mesh in some locations;

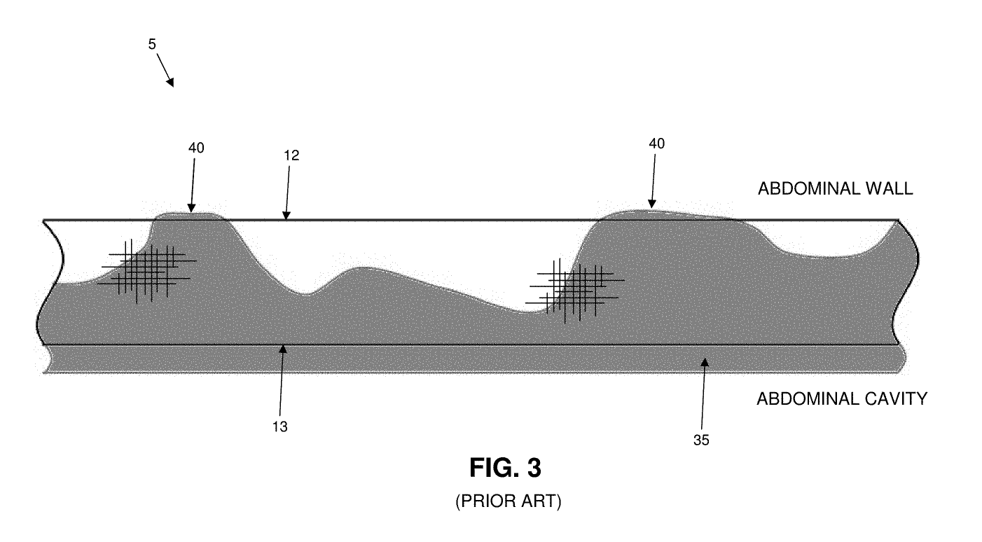

FIG. 4 is a schematic view showing a non-knitted/non-woven surgical mesh having an ingrowth-preventing coating on one side of the non-knitted/non-woven surgical mesh, wherein the ingrowth-preventing coating penetrates completely through the non-knitted/non-woven surgical mesh in some locations;

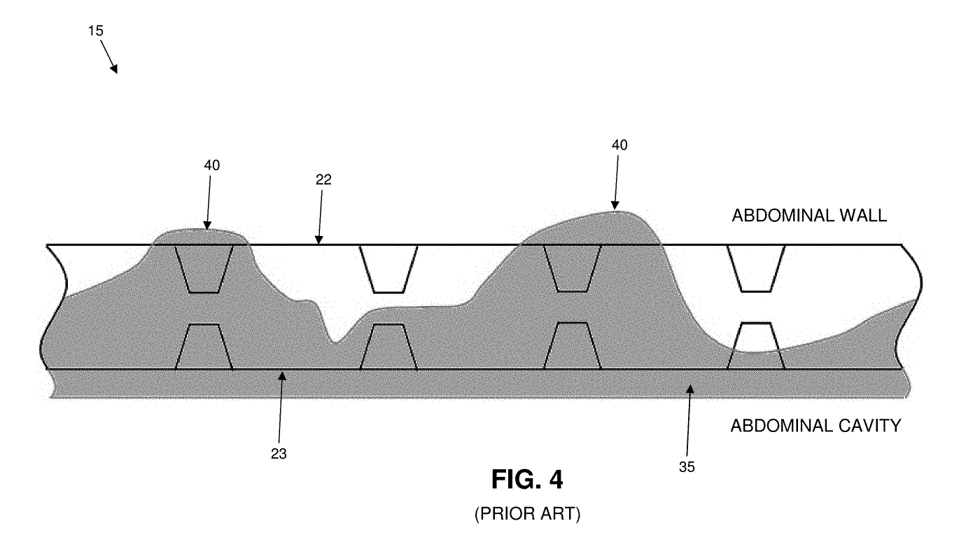

FIG. 5 is a schematic view showing a non-knitted/non-woven surgical mesh formed in accordance with the present invention, wherein an ingrowth-preventing coating is applied on one side of the non-knitted/non-woven surgical mesh and does not penetrate through to the other side of the non-knitted/non-woven surgical mesh;

FIG. 6 is a cross-sectional view of a finished example of a surgical mesh formed in accordance with the present invention, wherein a single pass silicone spray coverage with mechanical smoothing demonstrates sufficient penetration into the surgical mesh and excellent surface coverage with silicone but an unacceptably thick overall preparation with some surface holes;

FIG. 7 is a cross-sectional view of another finished example of a surgical mesh formed in accordance with the present invention, wherein a double pass silicone spray coverage demonstrates superior silicone penetration but insufficient surface coverage with silicone; and

FIG. 8 is a cross-sectional view of another finished example of a surgical mesh formed in accordance with the present invention, wherein a double pass silicone spray coverage demonstrates good silicone penetration and excellent surface coverage with minimal-to-no holes in the surface silicone coverage.

### DETAILED DESCRIPTION OF THE PREFERRED EMBODIMENTS

The present invention comprises a new and improved approach for applying an ingrowth-preventing coating (which is permanent or resorbable, and adhesion-preventing) on one side of a surgical mesh (e.g., on the abdominal cavity side of a surgical mesh) while ensuring that the ingrowth-preventing coating does not penetrate through to the other side of the surgical mesh (e.g., the abdominal wall side) of the surgical mesh.

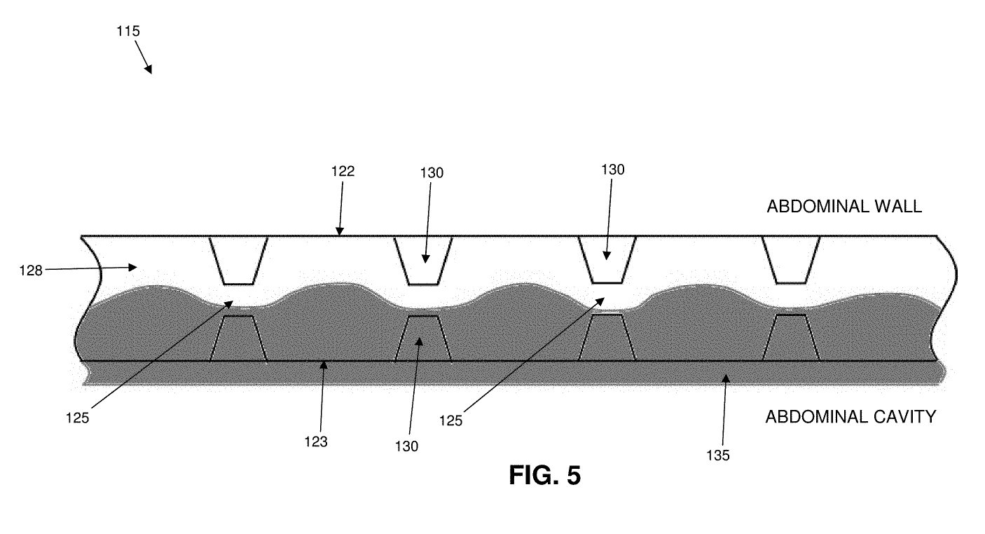

More particularly, and looking now at FIG. 5, there is shown a novel non-knitted/non-woven surgical mesh 115 formed in accordance with the present invention. In one preferred form of the invention, non-knitted/non-woven surgical mesh 115 comprises a matrix of polypropylene fibers fused together (e.g., at 125) by a compressive heat process so as to form a composite porous structure. In another preferred form of the invention, non-knitted/non-woven surgical mesh 115 comprises a matrix of polyester fibers fused together (e.g., at 125) by a compressive heat process so as to form a composite porous structure. In still another preferred form of the invention, non-knitted/non-woven surgical mesh 115 comprises a matrix of fibers formed out of another material (e.g., another polymer), with the fibers being fused together (e.g., at 125) by a compressive heat process so as to form a composite porous structure. In this respect it will be appreciated that the compressive heat fusion process typically leaves a plurality of open pores in the non-woven matrix and regularly-spaced recesses 130 which extend partially into, but not all of the way through, the thickness of the non-knitted/non-woven surgical mesh.

An ingrowth-preventing coating 135 formed out of a permanent or resorbable non-porous flexible coating (e.g., silicone, urethane, a resorbable material, etc.) is applied to second surface 123 of non-knitted/non-woven surgical mesh 115. In accordance with the present invention, ingrowth-preventing coating 135 does not penetrate through to the other side of the non-knitted/non-woven surgical mesh 115 (i.e., ingrowth-preventing coating 135 does not penetrate through to first surface 122 of non-knitted/non-woven surgical mesh 115). As a result, non-knitted/non-woven surgical mesh 115 permits tissue ingrowth into first surface 122 of non-knitted/non-woven surgical mesh 115 (e.g., into the abdominal wall side of the surgical mesh) and prevents tissue ingrowth into second surface 123 of non-knitted/non-woven surgical mesh 115 (e.g., into the abdominal cavity side of the surgical mesh).

Non-knitted/non-woven barrier surgical mesh 115 is manufactured by spraying ingrowth-preventing coating 135 onto second surface 123 of non-knitted/non-woven surgical mesh 115 (i.e., the abdominal cavity side of non-knitted/non-woven surgical mesh 115). More particularly, a liquid solution of silicone, having a desired degree of viscosity, is prepared, e.g., by thinning the silicone to an appropriate extent using an appropriate organic solvent such as hexane. In one preferred form of the present invention, the liquid solution of silicone (i.e., a diluted elastomer) comprises a mixture of 60% silicone and 40% hexane solvent. A spray technique is then used to apply the liquid silicone solution to second surface 123 of non-knitted/non-woven barrier surgical mesh 115 (e.g., on the abdominal cavity side of non-knitted/non-woven surgical mesh 115) in a controlled manner. The silicone solution penetrates the non-knitted/non-woven surgical mesh 115 to a desired extent, but in any case does not penetrate all the way through to first surface 122 of non-knitted/non-woven surgical mesh 115 (e.g., the abdominal wall side of the non-knitted/non-woven surgical mesh 115). In one preferred form of the present invention, ingrowth-preventing coating 135 penetrates 10-40% of the depth of non-knitted/non-woven surgical mesh 115. In another preferred form of the present invention, ingrowth-preventing coating 135 penetrates 40-60% of the depth of non-knitted/non-woven surgical mesh 115. In yet another preferred form of the present invention, ingrowth-preventing coating 135 penetrates 60-90% of the depth of non-knitted/non-woven surgical mesh 115. It should be appreciated that, inasmuch as compressed portions 125 of non-knitted/non-woven surgical mesh 115 have a higher density than the remainder of the surgical mesh, ingrowth-preventing coating 135 penetrates these areas to a lesser degree.

Ingrowth-preventing coating 135 is then cured so that the ingrowth-preventing coating 135 adheres to non-knitted/non-woven surgical mesh 115. Such curing is preferably achieved by the application of appropriate heat, UV irradiation, drying, a catalyst, gamma radiation, etc. In one form of the present invention, curing is achieved by a combination of the foregoing, e.g., by the application of appropriate heat and a catalyst.

Alternatively, the ingrowth-preventing coating may comprise a liquid solution of urethane having a desired degree of viscosity, e.g., by thinning the urethane to an appropriate extent using an appropriate solvent (e.g., hexane).

It should be appreciated that, by virtue of the foregoing approach, the degree to which the ingrowth-preventing coating 135 penetrates the non-knitted/non-woven surgical mesh 115 can be controlled by varying (i) the composition and construction of non-knitted/non-woven surgical mesh 115, (ii) the composition of ingrowth-preventing coating 135, and (iii) the spray and curing parameters of the deposition and setting processes of the ingrowth-preventing coating 135. In any case, ingrowth-preventing coating 135 forms a substantially complete coating on second surface 123 of the non-knitted/non-woven surgical mesh 115 (e.g., on the abdominal cavity side of the surgical mesh) and does not penetrate through to first surface 122 of non-knitted/non-woven surgical mesh 115.

It should also be appreciated that ingrowth-preventing coating 135 may be formed by resorbable elastomers well known in the art of polymer chemistry. By way of example but not limitation, ingrowth-preventing coating 135 may comprise at least one from the group consisting of polytrimethylcarbonate, polycaprolactone, polydioxanone and polybutyleneterephthalate. Additionally, the aforementioned resorbable polymers may also be mixed with other resorbable materials well known in the art such as polylactic acid (PLA) and/or polyglycolic acid (PGA) in order to optimize certain properties of the ingrowth-preventing coating (e.g., the strength of the coating, the stiffness of the coating, the resorption properties of the coating, etc.).

Thus it will be seen that the present invention may be used to provide a novel non-knitted/non-woven surgical mesh having a well controlled, substantially flat, tissue ingrowth-preventing coating on one side of the non-knitted/non-woven surgical mesh (e.g., the abdominal cavity side) and a porous, tissue ingrowth-promoting surface on the opposite side of the non-knitted/non-woven surgical mesh.

### EXAMPLES

The following examples demonstrate variation in four key variables that are critical in producing a satisfactory one-sided adhesion-reducing barrier mesh. The intent of the process is to produce a one-sided adhesion barrier without substantially increasing the overall thickness of the barrier mesh over the base sheet material thickness, ensuring sufficient barrier penetration into the base material for good mechanical integration, and leaving the majority of the base material matrix void space available for tissue integration.

### Example 1

A sheet of 0.5 mm non-knitted/non-woven polypropylene material 80 g/m² in weight is spray coated with two part heat- and catalyst-cured flowable silicone elastomer NuSil LSR-4850 thinned with the organic solvent hexane to achieve a volume mixture of 60% silicone and 40% hexane solvent on one surface. The silicone/hexane mixture (hereinafter sometimes referred to as “the silicone coating”) is sprayed to achieve complete surface coverage of the non-knitted/non-woven material surface. A straight edge is taken and any surface irregularities of the silicone coating smoothed to ensure complete silicone coverage of the surface, no holes in the silicone coverage and a smooth silicone surface.

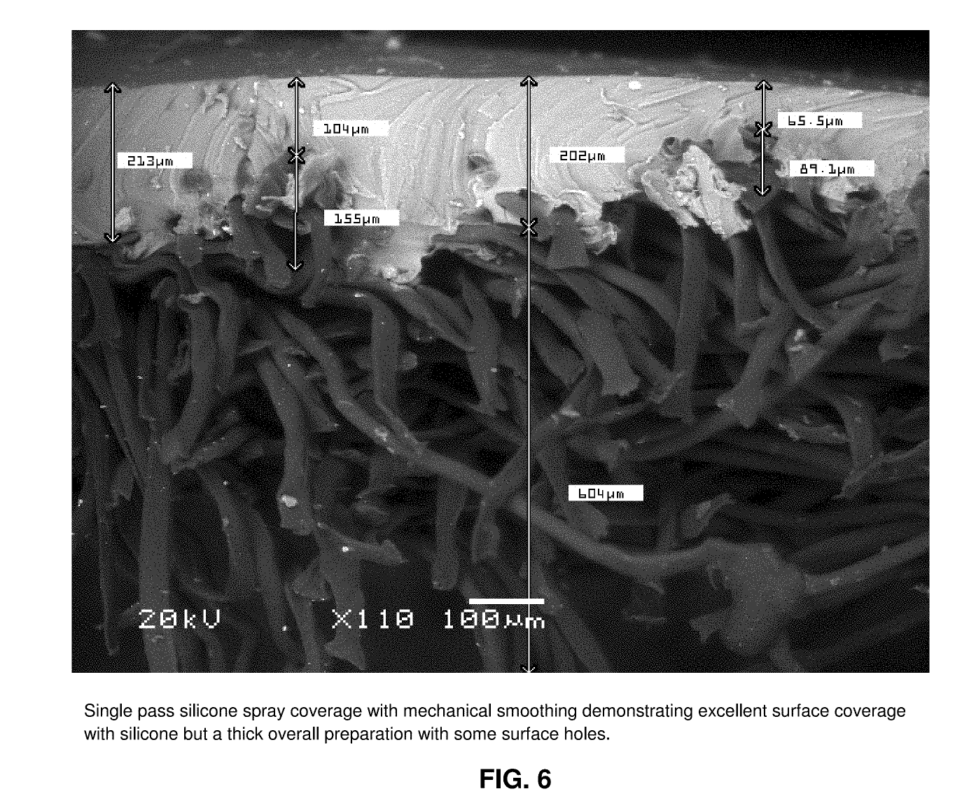

This process approach achieves a coated non-knitted/non-woven barrier mesh of 0.85 mm thickness with silicone alone constituting 12% of the thickness, silicone and non-knitted/non-woven polypropylene constituting 18% of the thickness and non-knitted/non-woven polypropylene constituting 71% of the total barrier mesh thickness. The resulting barrier mesh sample is thick with good surface coverage, minimal surface holes, good penetration of the silicone into the non-knitted/non-woven structure and excellent availability of the non-knitted/non-woven structure for tissue integration. FIG. 6 shows the cross-section of the finished example barrier mesh.

### Example 2

A sheet of 0.5 mm non-knitted/non-woven polypropylene material 80 g/m² in weight is spray coated on one surface with the two part heat- and catalyst-cured flowable silicone elastomer NuSil LSR-4850 thinned with the organic solvent hexane to achieve a volume mixture of 50% silicone and 50% hexane solvent. The silicone/hexane mixture is sprayed to achieve complete surface coverage of the non-knitted/non-woven material surface.

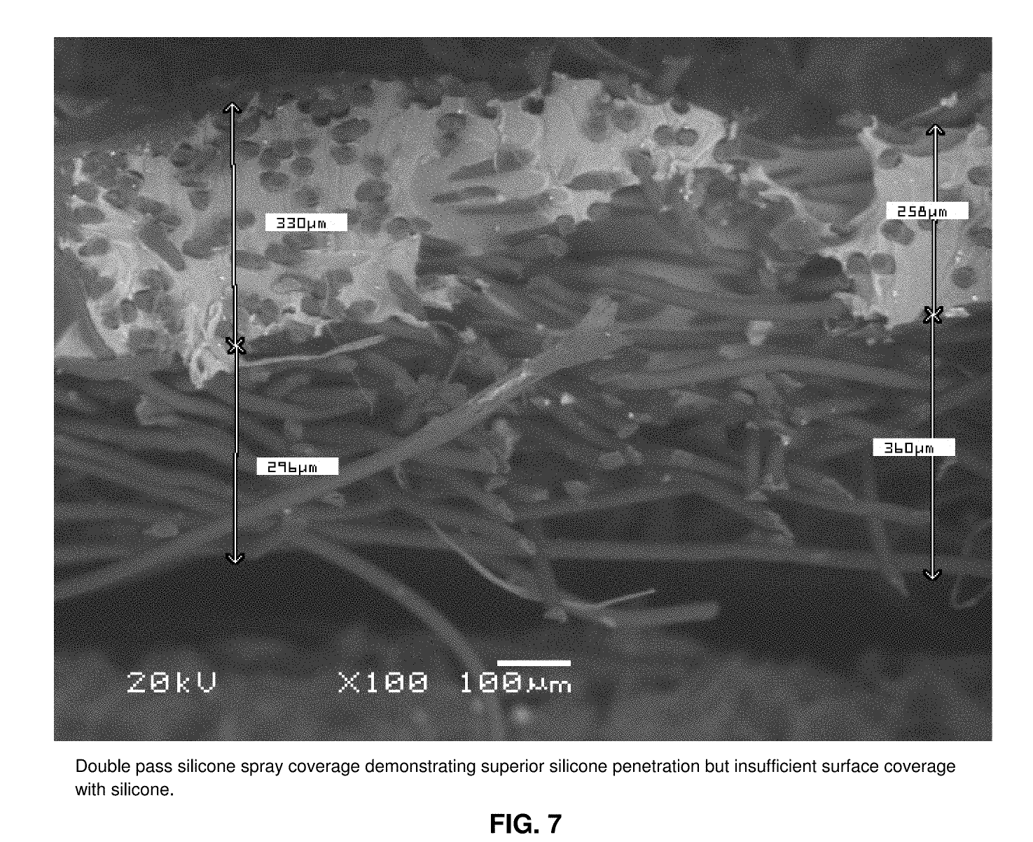

This process achieves a one surface coated non-knitted/non-woven barrier mesh of 0.69 mm thickness with silicone alone constituting 0% of the thickness, silicone and non-knitted/non-woven polypropylene constituting 48% of the thickness and non-knitted/non-woven polypropylene constituting 52% of the total barrier mesh thickness. The resulting barrier mesh sample is thinner with poor surface coverage, a plethora of surface holes, excellent penetration of the silicone into the non-knitted/non-woven polypropylene so that separation of the two components would be unlikely, and a substantial portion of the non-knitted/non-woven structure still available for tissue integration. FIG. 7 shows the cross-section of the finished example barrier mesh.

### Example 3

A sheet of 0.5 mm non-knitted/non-woven polypropylene material 80 g/m² in weight is spray coated with two applications of the two part heat- and catalyst-cured flowable silicone elastomer NuSil LSR-4850 thinned with the organic solvent hexane to achieve a volume mixture of 40% silicone and 60% hexane solvent on one surface. The silicone/hexane mixture is sprayed to achieve complete surface coverage of the non-knitted/non-woven material surface.

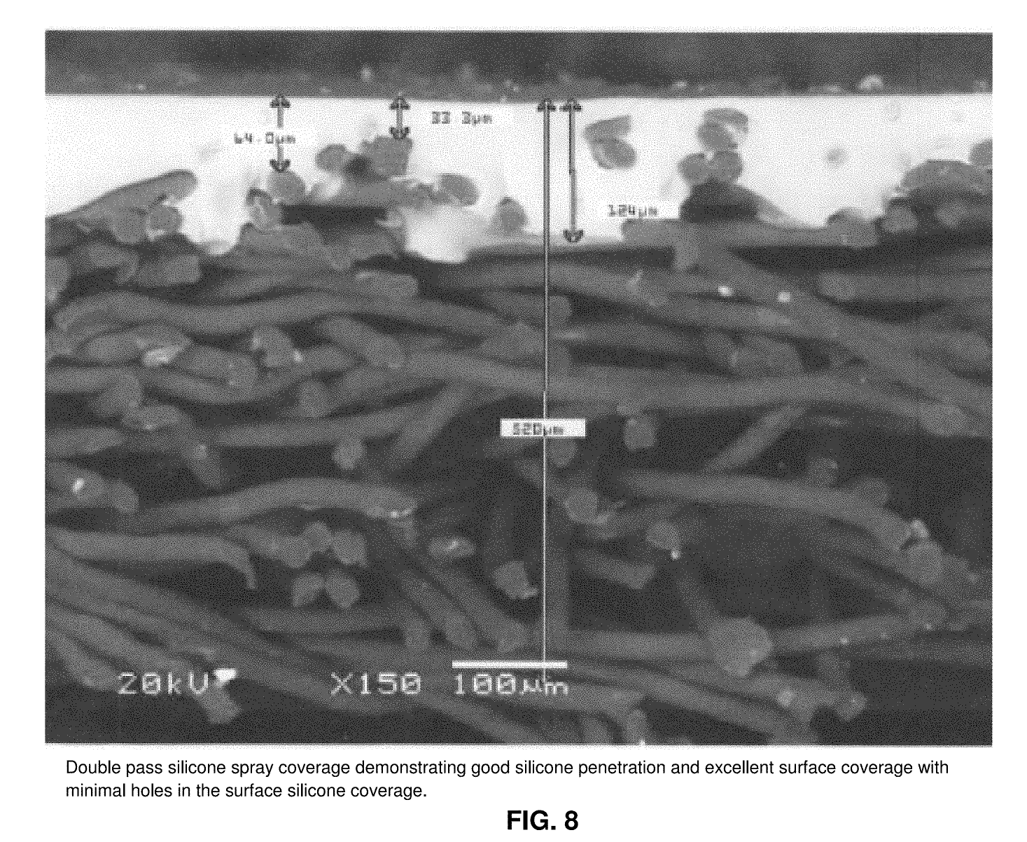

This process approach achieves a one surface coated non-knitted/non-woven barrier mesh of 0.52 mm thickness with silicone alone constituting 7% of the thickness, silicone and non-knitted/non-woven polypropylene constituting 9% of the thickness and non-knitted/non-woven polypropylene constituting 83% of the total barrier mesh thickness. The resulting barrier mesh sample is thinner with excellent surface coverage and few surface holes. The penetration of the silicone into the non-knitted/non-woven polypropylene is minimal so that separation of the two components could be expected. FIG. 8 shows the cross-section of the finished example barrier mesh.

### Use of the Present Invention with Knitted/Woven Surgical Meshes

It should be appreciated that the present invention may also be utilized with knitted/woven surgical meshes. In this case, the ingrowth-preventing coating (e.g., a permanent or resorbable non-porous flexible coating such as silicone, urethane, a flexible resorbable material, etc., preferably thinned with an appropriate solvent) is applied with a spray technique to the second surface of the knitted/woven surgical mesh and then cured, so that the ingrowth-preventing coating forms a substantially complete coating on the second surface of the knitted/woven surgical mesh (e.g., on the abdominal cavity side of the knitted/woven surgical mesh) and does not penetrate through to the first surface of the knitted/woven surgical mesh.

### MODIFICATIONS OF THE PREFERRED EMBODIMENTS

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A surgical mesh, said surgical mesh comprising:
a matrix of fibers, said matrix comprising a first surface and a second surface; and
a tissue ingrowth-preventing material applied to said second surface of said matrix;
wherein said tissue ingrowth-preventing material penetrates only part way through said matrix.

2. A surgical mesh according to claim 1 wherein said fibers have a random orientation.

3. A surgical mesh according to claim 1 wherein said fibers have an ordered orientation.

4. A surgical mesh according to claim 1 wherein said matrix of fibers comprises a flexible, biocompatible material.

5. A surgical mesh according to claim 1 wherein said matrix of fibers is resorbable.

6. A surgical mesh according to claim 1 wherein said matrix of fibers is non-resorbable.

7. A surgical mesh according to claim 1 wherein said matrix of fibers comprises a non-knitted/non-woven material.

8. A surgical mesh according to claim 7 wherein said non-knitted/non-woven material comprises a polymer.

9. A surgical mesh according to claim 8 wherein said non-knitted/non-woven material comprises polypropylene.

10. A surgical mesh according to claim 8 wherein said non-knitted/non-woven material comprises fluorinated polyolefin.

11. A surgical mesh according to claim 7 wherein said non-knitted/non-woven material comprises a ceramic.

12. A surgical mesh according to claim 7 wherein said non-knitted/non-woven material comprises a metal.

13. A surgical mesh according to claim 1 wherein said matrix of fibers comprises a knitted/woven material.

14. A surgical mesh according to claim 13 wherein said knitted/woven material comprises polypropylene.

15. A surgical mesh according to claim 13 wherein said knitted/woven material comprises polyester.

16. A surgical mesh according to claim 1 wherein said tissue ingrowth-preventing material comprises a curable, flexible, biocompatible material.

17. A surgical mesh according to claim 1 wherein said tissue ingrowth-preventing material is resorbable.

18. A surgical mesh according to claim 1 wherein said tissue ingrowth-preventing material is non-resorbable.

19. A surgical mesh according to claim 1 wherein said tissue ingrowth-preventing material comprises an elastomer.

20. A surgical mesh according to claim 19 wherein said tissue ingrowth-preventing material comprises silicone.

21. A surgical mesh according to claim 19 wherein said tissue ingrowth-preventing material comprises urethane.

22. A surgical mesh according to claim 1 wherein said tissue ingrowth-preventing material is applied to said second surface as a liquid.

23. A surgical mesh according to claim 22 wherein said tissue ingrowth-preventing material cures to a flexible solid.

24. A surgical mesh according to claim 1 wherein said tissue ingrowth-preventing material penetrates 10-40% of the depth of said matrix of fibers.

25. A surgical mesh according to claim 1 wherein said tissue ingrowth-preventing material penetrates 40-60% of the depth of said matrix of fibers.

26. A surgical mesh according to claim 1 wherein said tissue ingrowth-preventing material penetrates 60-90% of the depth of said matrix of fibers.

27. A method of manufacturing a surgical mesh, said method comprising:
providing a matrix of fibers, said matrix comprising a first surface and a second surface; and
spraying a tissue ingrowth-preventing material onto said second surface of said matrix such that said tissue ingrowth-preventing material penetrates into said matrix part way, but not all of the way, through said matrix.

28. A method according to claim 27 further comprising the step of curing said tissue ingrowth-preventing material.

29. A method according to claim 28 wherein the step of curing comprises curing with ultraviolet light.

30. A method according to claim 28 wherein the step of curing comprises curing with a catalyst.

31. A method according to claim 28 wherein the step of curing comprises curing with heat.

32. A method according to claim 28 wherein the step of curing comprises curing with gamma radiation.

33. A method according to claim 28 wherein the step of curing comprises curing with drying.

34. A method according to claim 28 wherein the step of curing comprises curing with both heat and a catalyst.
